(19) United States
(12) Patent Application Publication
(10) Pub. No.: US 2023/0088479 A1
(43) Pub. Date: Mar. 23, 2023

(54) VEHICLE WITH MODULAR AIR TREATMENT UNIT

(71) Applicant: Renewed Performance Company, Inc., Tiptpn, IN (US)

(72) Inventor: Christopher A. Palabrica, Tipton, IN (US)

(21) Appl. No.: 17/950,537

(22) Filed: Sep. 22, 2022

Related U.S. Application Data
(60) Provisional application No. 63/246,818, filed on Sep. 22, 2021.

(51) Int. Cl. A61L 9/20; B60H 3/00; B60H 3/06

(52) U.S. Cl. CPC A61L 9/20; B60H 3/0078; B60H 3/0608; A61L 2209/134; A61L 2209/16; A61L 2209/14

(57) ABSTRACT

A modular air treatment unit for an over the road vehicle is independent of an associated climate control system. The air treatment unit is configured to filter and sanitize air within the passenger compartment as air moves along a flow path separate from a flow path through the climate control system. The modular air treatment unit includes a housing defining a circuitous passageway, an air filter mounted to the housing, a fan configured to move air along passageway, and an ultraviolet light source arranged in the housing. The ultraviolet light source is configured to discharge germicidal ultraviolet light inside the housing to eliminate pathogens in the housing.

### PRIORITY CLAIM

This application claims priority under 35 U.S.C. § 119(e) to U.S. Provisional Application Ser. No. 63/246,818, filed Sep. 22, 2021, which is expressly incorporated by reference herein.

### BACKGROUND

Air quality within vehicles is an area of continued development, especially in view of disease-spreading pathogens and/or cancer-causing carcinogens that can be deposited in a vehicle during mass transit use or during specialty vehicle use. For example, buses can carry hundreds of passengers per day who can deposit pathogens in the bus. Also, ambulances carry patients (and caregivers) who can deposit pathogens potentially very harmful to future passengers. In another example, firetrucks can be contaminated with unwanted pathogens, chemicals, or carcinogens from fire events.

### SUMMARY

A modular air treatment unit for vehicles that can be retrofitted as an aftermarket improvement is disclosed in this paper. The air treatment unit is independent of an associated climate control system for ease of aftermarket installation. The air treatment unit is configured to filter and sanitize air within the passenger compartment of the vehicle as air moves along a flow path separate from a flow path through the climate control system to support health and safety of vehicle occupants.

In illustrative embodiments, the modular air treatment unit includes a housing, an air filter mounted to the housing, a fan configured to move air through the filter and housing, and an ultraviolet light source arranged in the housing. The ultraviolet light source is configured to discharge germicidal, UV-C ultraviolet light inside the housing to eliminate pathogens in the housing.

In illustrative embodiments, the housing defines a circuitous passageway between an inlet and an outlet to extend the distance air travels through the housing where the air interacts with germicidal ultraviolet light. In addition, the exemplary housing is made from an aluminum or other suitable material with reflectivity that reflects the germicidal ultraviolet light to increase interaction with air in the housing.

In illustrative embodiments, light barriers are arranged at the inlet and outlet of the housing to discourage the germicidal ultraviolet light from leaving the housing. In some examples, air filters provide one or more of the light barriers. In this way the ultraviolet light is contained in the housing and occupants of the passenger compartment avoid interaction with the ultraviolet light.

In illustrative embodiments, the fan and ultraviolet lights of the modular air treatment unit are configured to be powered by batteries included in the vehicle. More specifically, all components of the modular air treatment unit may be configured to be powered by 12 volt direct current rather than by other power sources such as an internal combustion engine drive or high-voltage battery adapted for electric propulsion of the vehicle.

Additional features of the present disclosure will become apparent to those skilled in the art upon consideration of illustrative embodiments exemplifying the best mode of carrying out the disclosure as presently perceived.

### BRIEF DESCRIPTIONS OF THE DRAWINGS

The detailed description particularly refers to the accompanying figures in which:

FIG. 1 is a partially diagrammatic view of a bus configured for mass transit showing that the bus includes a vehicle body defining a passenger compartment, a climate control system for heating/cooling the passenger compartment, and a modular air treatment unit independent of the climate control system for filtering and sanitizing air within the passenger compartment to protect the health of vehicle occupants;

FIG. 2 is a front perspective view of the modular air treatment unit showing an inlet into a housing of the modular air treatment unit;

FIG. 3 is a rear perspective view of the modular air treatment unit showing an outlet out of the housing of the modular air treatment unit;

FIG. 4 is a side elevation view of the modular air treatment unit showing that the modular air treatment unit includes a fan and a filter mounted at the inlet of the housing;

FIG. 5 is a plan view of the modular air treatment unit showing that the housing includes internal walls defining a circuitous, serpentine passageway from the inlet to an outlet, and showing ultraviolet LED strips arranged through the passageway to discharge germicidal ultraviolet light in the passageway that interacts with air moving through the modular air treatment unit so as to sanitize the air prior to exiting the housing;

FIG. 6 is a top perspective view of the modular air treatment unit with a lid removed from the housing to expose the fan, the passageway through the housing, and the ultraviolet LED strips arranged through the passageway;

FIG. 7 is another top perspective view of the modular air treatment unit with a lid removed showing the inlet and fan; and

FIG. 8 is a exploded perspective assembly view of the modular air treatment unit shown in FIGS. 6 and 7.

### DETAILED DESCRIPTION

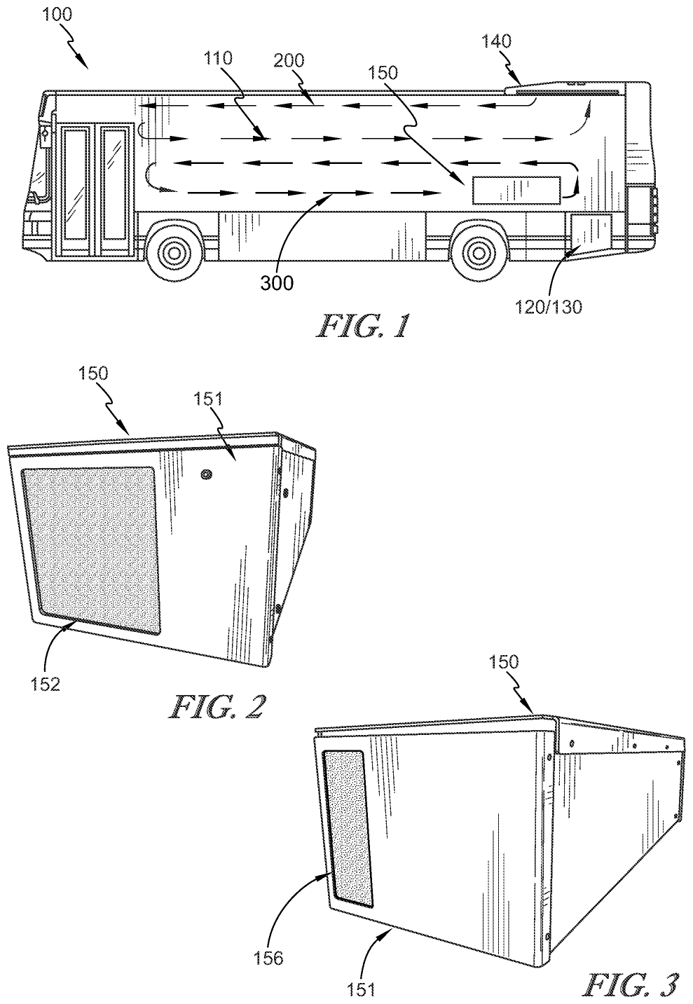

An over the road vehicle, illustrated as a bus, includes a vehicle body 100 defining a passenger compartment 110, a climate control system 140, and a modular air treatment unit 150 independent of the climate control system 140 as shown in FIG. 1. The climate control system 140 and the modular air treatment unit 150 both distribute air in the passenger compartment 110 but flow air through two separate flow paths 200/300. The climate control system 140 distributes heated or cooled air, defined by a first flow path 200, for passenger comfort while the modular air treatment unit 150 filters air, defined by a second flow path 300. The modular air treatment unit 150 sanitizes air flowing through this second flow path 300 to reduce risks from pathogens, carcinogens, and other unwanted contaminants without reliance on the climate control system 140.

The climate control system 140 is configured to heat and cool the passenger compartment 110 through the first flow path 200, as shown in FIG. 1. The climate control system 140 includes thermal elements, an air mover, and ducts fluidly coupling the thermal elements to the passenger compartment. In the illustrated design, an internal combustion engine 120 provides a heated thermal element and an air conditioning compressor provides a cooled thermal element. The air mover is configured to move air into thermal communication with one of the thermal elements. The ducts carry heated/cooled air to one or more discharge vents opening into the passenger compartment. In other embodiments, thermal elements may be electrically provided via resistors, thermal electric devices (TEDs), and/or other suitable systems.

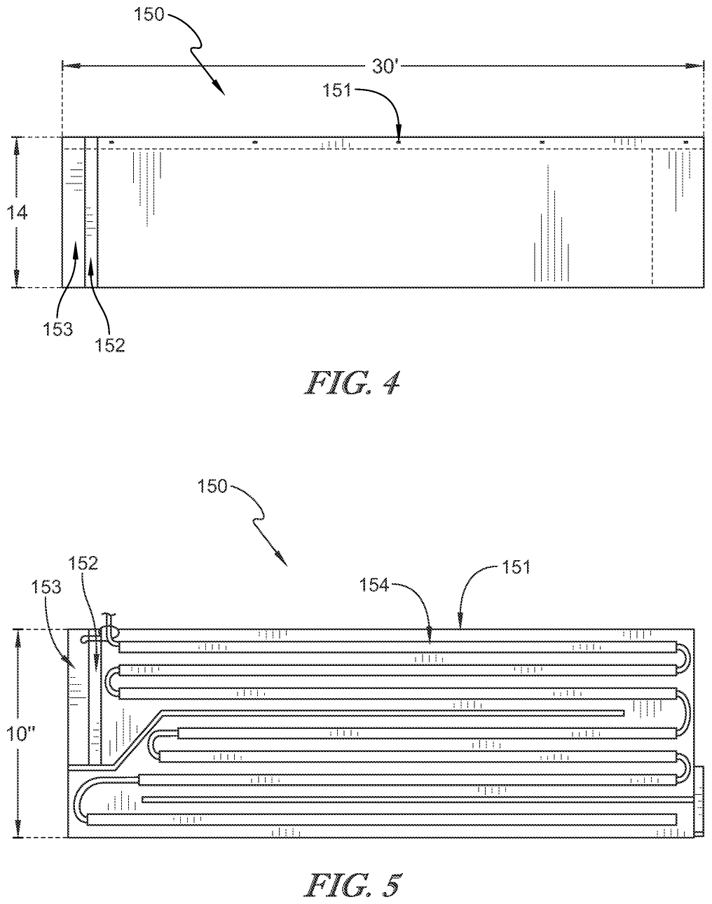

The modular air treatment unit 150 is separate from the climate control system 140 to allow for aftermarket installation without complex or costly integration with the climate control system 140 as suggested in FIG. 1. The modular air treatment unit 150 illustratively includes a housing 159, a fan 153, air filters 156, and LED strips 154 which emits ultraviolet light as shown in FIGS. 2-6. The housing 159 creates an interior circuitous, serpentine passageway 158 defining the second flow path 300 along which air is moved and exposed to ultraviolet light emitted from the LED strips 154 to kill pathogens.

Unlike standard climate control systems 140 which minimize the amount of turns in the system to avoid pressure drops in the travelling air, the modular air treatment unit 150 of the present disclosure aims to increase the number of turns. As shown in FIG. 5, the passageway 158 of the modular air treatment unit 150 consists of several 180 degree turns which increase the distance needed for air to travel from the inlet 151 to the outlet 152 and as such, increasing the surface area of air coming in contact with the ultraviolet light emitted from the LED strips 154.

The fan 153 moves air through the housing 159 and is configured to operate on 12 volts DC as is typically available from automotive batteries 130 without operation of an associated engine 120. The filters 156 catch dust and debris of small scale in order to cleanse air of large pathogens, particulate cancer-causing carcinogens, and unwanted dirt. The ultraviolet lights discharge UV-C type light that is germicidal and known to eliminate airborne pathogens.

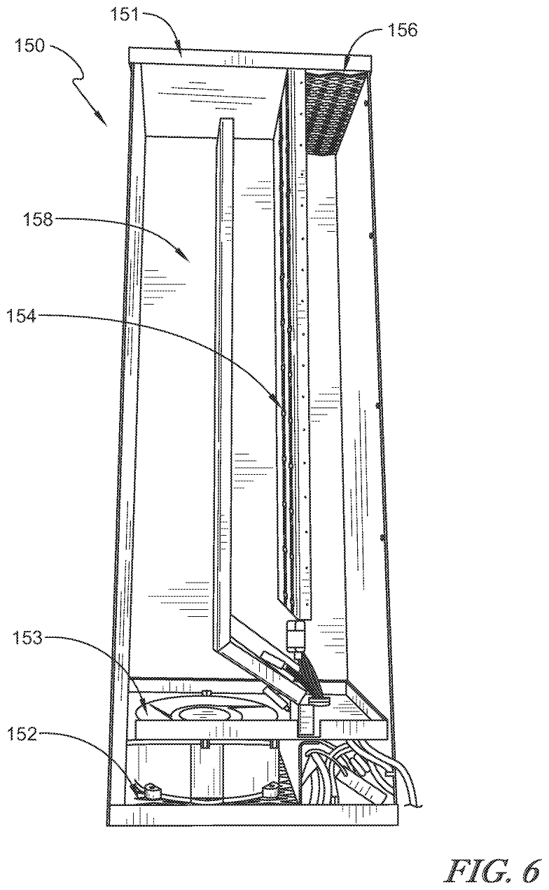

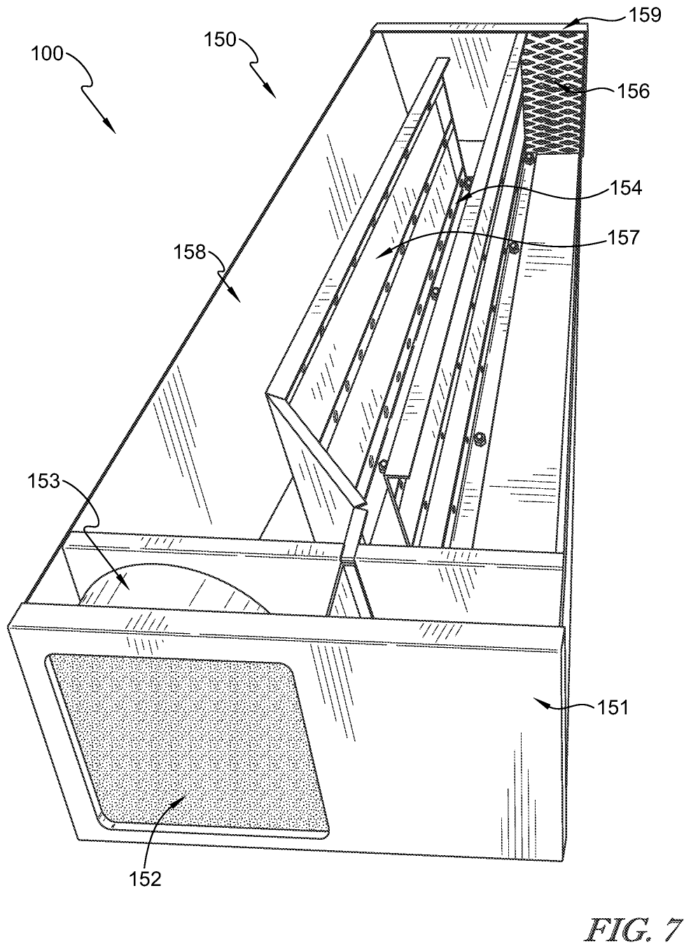

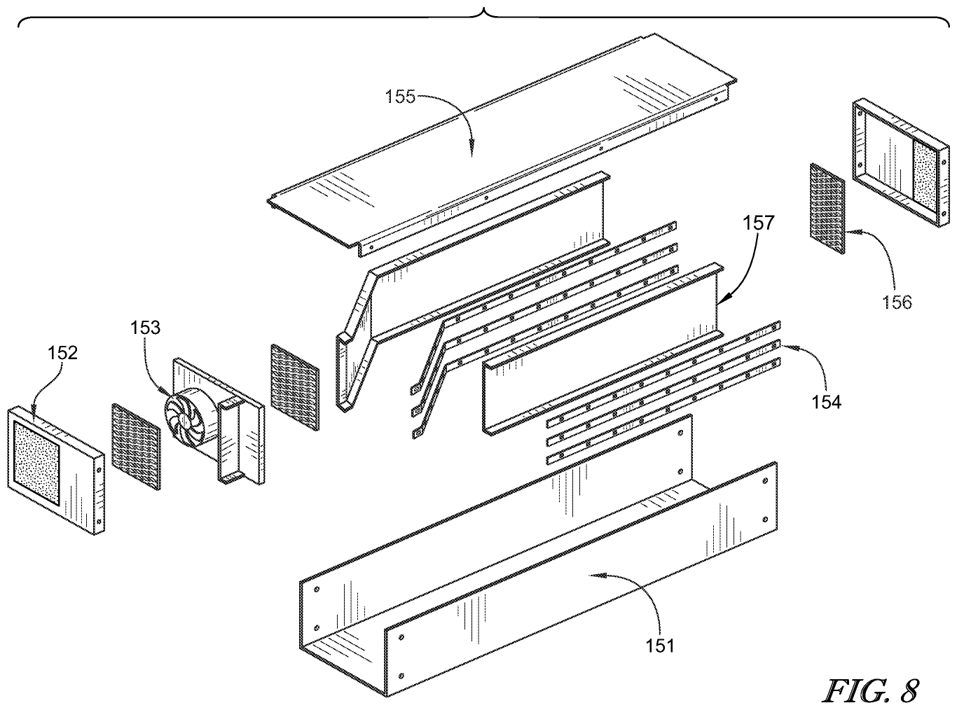

In the illustrated embodiment, the housing 159 is mounted in the passenger compartment 110 with its inlet and outlet in direct fluid communication with air in the passenger compartment 110. The circuitous passageway defined by the housing 159 between the inlet 151 and the outlet 152 extends the distance air and potential pathogens must travel while being exposed to ultraviolet light increasing the chance for pathogen destruction. The housing 159 is manufactured from dual action sanded (DA) aluminum with the reflective surface finish facing the internal walls 157 of the housing 159 as suggested in FIG. 6. Internal walls 157 facing the circuitous passageway have a reflectivity greater than 60 percent and up to 90+ percent to encourage reflection of the germicidal ultraviolet light around the passageway 158 for interaction with air moving through the passageway 158 defined by the second flow path 300.

The filters 156 in the example shown are placed at the inlet 151 and the outlet 152 of the housing 159 to interact with air passing into and out of the modular air treatment unit 150 through the second flow path 300. The filters 156 also serve as light barriers at the inlet 151 and the outlet 152 to discourage egress of the ultraviolet light emitted by the LED strips 154. The opaque housing 159, the lid 155, the internal walls 157 and the filters 156 at the inlet 151 and the outlet 152 all function as light barriers to block between 85% to 99% of all ultraviolet light from escaping the modular air treatment unit 150 into the passenger compartment 110. This helps to protect the vehicle 100 interior and occupants in the passenger compartment 110 from possibly harmful exposure to ultraviolet light.

UV-C light as is discharged by the LED strips 154 to provide germicidal effect can prematurely fade the interior of the vehicle 100, cause cracking, and even lead to skin/eye injuries to exposed individuals. Accordingly, containing the UV-C light in the housing 159 can be a desirable feature allowing use of the modular air treatment unit 150 while occupants are in the vehicle 100.

The fan 153 of the shown design is mounted in a plate at the inlet 151 and provides further light barrier discouraging ultraviolet light egress. In the illustrative embodiment, the filters 156 are configured to block substantially all ultraviolet light from escaping the housing 159.

The ultraviolet lights in the disclosed embodiment are produced by light emitting diodes (LED strips 154) configured to discharge ultraviolet light with a generally, or precisely, 270-280 nanometer wavelength. The LED strips 154 are provided in strings powered by a 12 volt DC power source, such as a standard vehicle battery 130. The LED strips 154 are arranged and fixed to the internal walls 157 of the housing 159 along the passageway 158 between the inlet 151 and the outlet 152. In other embodiments, other sources of germicidal ultraviolet light may be used inside the housing 159.

In some embodiments, the modular air treatment unit 150 may include an external light (LED) that illuminates to confirm the modular air treatment unit 150 is in operation. In other embodiments, other indicators may be used to confirm operation of the modular air treatment unit 154 since operation of the fan 153 and the LED strips 154 may be visually/audibly undetectable outside the housing 159.

The modular air treatment unit 150 of the present disclosure is adapted for simple integration into the accessory 12 volt DC circuit powered by a vehicle battery 130. In this embodiment, the modular air treatment unit 150 is powered and operates anytime the vehicle 100 is operating or in accessory power mode. In exemplary designs, the modular air treatment unit 150 may be mounted under a seat or to a ceiling of the vehicle 100. However, other locations in which the modular air treatment unit 150 may have direct fluid communication with the passenger compartment 110 are also contemplated. The presently disclosed design facilitates aftermarket integration at low cost while providing high value air treatment for occupants of the vehicle 100.

In some embodiments, the modular air treatment unit 150 may be directly coupled to a vehicle battery 130, a separate, dedicated battery, or another power source. The modular air treatment unit 150 may incorporate a controller (processor and memory with instructions) or integrate with a controller for the vehicle 100 configured to direct operation of the modular air treatment unit 150. In embodiments with a controller, operation of the modular air treatment unit 150 may be optimized for a particular application.

In one example of a controller-enabled modular air treatment unit 150 used in a vehicle 100, such as a bus, the controller may be configured to operate before and/or after planned use of the vehicle 100. Specifically, the controller may operate the modular air treatment unit 150 before and/or after the end of a day's routes to filter and sanitize air on the bus, defined by the second flow path 300, when all occupants out of the passenger compartment 110. Such operation may be in addition to continuous or intermittent use during operation of the vehicle 100 based on various factors. In certain embodiments, the controller may operate the modular air treatment unit 150 during operation/non-operation of the vehicle 100 based at least in part on the volumetric size of the passenger compartment 110, a detected charge of the battery 130 (i.e. if the battery 130 is adequately charged for use while the vehicle 100 is not in operation), schedule/location/route data, air quality data (i.e. particulate levels), time of the year (i.e. flu season), general/localized health data (i.e. infection rates), environmental data (i.e. air quality), weather data (i.e. conditions suitable for pathogen spread), and/or other factors.

In another example, a controller-enabled modular air treatment unit 150 may be used in an ambulance where the controller may be configured to operate the modular air treatment unit 150 before and/or after use of the ambulance. Specifically, the controller may operate the modular air treatment unit 150 before and/or after a planned or emergent patient run to filter and sanitize air on the ambulance. Such operation may be in addition to continuous or intermittent use during operation of the vehicle 100 based on various factors. In certain embodiments, the controller may operate the modular air treatment unit 150 during operation/non-operation of the vehicle 100 based at least in part on the volumetric size of the passenger compartment 110, a detected charge of the battery 130 (i.e. if the battery 130 is adequately charged for use while the vehicle 100 is not in operation), schedule/location/route data, air quality data (i.e. particulate levels), time of the year (i.e. flu season), general/localized health data (i.e. infection rates), environmental data (i.e. air quality), weather data (i.e. conditions suitable for pathogen spread), patient-specific data (i.e. was a recent patient carrying a known pathogen, is an expected patient carrying a known pathogen, is an expected patient particularly vulnerable to pathogens that may be eliminated by modular air treatment unit 150, etc.) and/or other factors.

In yet another example of a controller-enabled modular air treatment unit 150 used in a firetruck, the controller may be configured to operate before and/or after use of the firetruck. Specifically, the controller may operate the modular air treatment unit 150 before and/or after a planned or emergent fire run to filter and sanitize air in the firetruck. Such operation may be in addition to continuous or intermittent use during operation of the vehicle 100 based on various factors. For instance, firetrucks are often idle for long periods between runs. In certain embodiments, the controller may operate the modular air treatment unit 150 multiple times between runs to filter and sanitize air in the passenger compartment 110. In addition, the controller may be configured to operate for a period after the passenger compartment 110 is exposed (or likely exposed) to carcinogenic materials as determined by user inputs, run type, location served, functions of the firetruck used, or the like. Moreover, the modular air treatment unit 150 may be operated during operation/non-operation of the vehicle 100 based at least in part on the volumetric size of the passenger compartment 110, a detected charge of the battery 130 (i.e. if the battery 130 is adequately charged for use while the vehicle 100 is not in operation), schedule/location/route data, air quality data (i.e. particulate levels), time of the year (i.e. flu season), general/localized health data (i.e. infection rates), environmental data (i.e. air quality), weather data (i.e. conditions suitable for pathogen spread), and/or other factors.

While shown and described in the context of an over the road vehicle 100, it is contemplated that the disclosed modular air treatment unit 150 may be used in other vehicles such as boats, trains, and/or planes. In each of these applications, addition of an independent modular air treatment unit 150 for air filtering and sanitation provides a straightforward solution for aftermarket upgrade. Such an upgrade avoids complex integration with existing climate control systems 140 and keeps potentially harmful ultraviolet light, emitted from the LED strips 154, from affecting occupants. This objective is further achieved with a low, managed power draw having negligible effects on efficiency of the vehicle 100.

It is contemplated that the modular air treatment unit 150 of the present disclosure may be configured for use with alternating current. For example if fitted in food trucks, blood donation busses, bookmobiles, motor coaches or the like, it may be desirable to power the unit via AC power available from onboard generators that provide power while the vehicle 100 is not in motion.

Moreover, it is contemplated that the modular air treatment unit 150 may be implemented in applications outside vehicles 100 and powered by power grids. For example, modular air treatment units 150 in line with the present disclosure can be used in individual offices, patient rooms, classrooms, homes, bathrooms and other locations. In these applications, the modular air treatment unit 150 is independent of building climate control systems 140. In this way, the modular air treatment unit 150 can be easily retrofitted in desired locations without disturbing the operation of existing climate control systems 140.

It is also contemplated that an air treatment system made up of a number of modular air treatment units 150 may be used to scale the benefit of the described modular air treatment unit 150. The number and location of individual modular air treatment units 150 may be determined based on the size and layout of a vehicle 100 or space to be filtered/sanitized. Operation of the modular air treatment units 150 in such a system may be coordinated based on various parameters, including those listed above for specific control of the vehicle.

1. An over the road vehicle, the vehicle comprising
a vehicle body defining passenger compartment,
a climate control system configured to heat and/or cool the passenger compartment, the climate control system including a thermal element, an air mover configured to move air across the thermal element, and a duct configured to carry air moved across the thermal element to one or more discharge vents into the passenger compartment, and
a modular air treatment unit independent of the climate control system that is configured to filter and sanitize air within the passenger compartment, the modular air treatment unit including a housing defining a circuitous passageway between an inlet in direct fluid communication with the passenger compartment and an outlet in direct fluid communication with the passenger compartment, an air filter mounted to the housing in fluid communication with the circuitous passageway, a fan configured to move air along the circuitous passageway from the inlet to the outlet, and an ultraviolet light source arranged in the housing and configured to discharge germicidal ultraviolet light inside the housing to interact with air moving along the circuitous passageway thereby killing pathogens in the housing.

2. The vehicle of claim 1, wherein the housing comprises interior surfaces facing the circuitous passageway with a reflectivity of greater than 70 percent to encourage reflection of the germicidal ultraviolet light around the circuitous passageway for interaction with air moving along the circuitous passageway.

3. The vehicle of claim 2, wherein the housing comprises at least one exterior surface opposing at least one of the interior surfaces facing the circuitous passageway having a reflectivity less than that of the at least one of the interior surfaces.

4. The vehicle of claim 2, wherein the housing is opaque and light barriers are arranged at the inlet and the outlet to discourage egress of the germicidal ultraviolet light and avoid interaction of the germicidal ultraviolet light with the passenger compartment thereby protecting occupants in the passenger compartment from unwanted exposure to ultraviolet light.

5. The vehicle of claim 4, wherein the light barriers are configured to retain at least 85% of the germicidal ultraviolet light inside the housing.

6. The vehicle of claim 4, wherein the air filter is arranged at the inlet and/or the outlet to provide at least a portion of one of the light barriers that discourage egress of the germicidal ultraviolet light from the housing.

7. The vehicle claim 6, wherein the fan is arranged at the inlet and/or the outlet to provide at least a portion of one of the light barriers that discourage egress of the germicidal ultraviolet light from the housing.

8. The vehicle of claim 1, wherein the fan and the ultraviolet light source are configured to operate on 12 V DC power available from a battery included in the vehicle.

9. The vehicle of claim 1, wherein the ultraviolet light source is provided by a series of LEDs in strips that extend along the circuitous passageway.

10. The vehicle of claim 9, wherein the ultraviolet light source is configured to discharge UV-C light with a 270-280 nanometer wavelength.

11. The vehicle of claim 1, wherein the modular air treatment unit includes an indicator light that is illuminated during operation.

12. The vehicle of any other claim or combination of claims, wherein the air filter is received in a slot defined by the housing so that the air filter is removable from the modular air treatment unit for cleaning or replacement.

13. A modular air treatment unit configured to filter and sanitize air within the passenger compartment of a vehicle, the unit comprising
a housing defining a circuitous passageway between an inlet configured for direct fluid communication with the passenger compartment and an outlet configured for direct fluid communication with the passenger compartment,
a fan configured to move air along the circuitous passageway from the inlet to the outlet,
an ultraviolet light source arranged in the housing and configured to discharge germicidal ultraviolet light inside the housing to interact with air moving along the circuitous passageway thereby eliminating pathogens in the housing, and
light barriers are arranged at the inlet and the outlet to discourage egress of the germicidal ultraviolet light and avoid interaction of the germicidal ultraviolet light with the passenger compartment thereby protecting occupants in the passenger compartment from unwanted exposure to ultraviolet light.

14. The unit of claim 13, wherein the housing comprises interior surfaces facing the circuitous passageway with a reflectivity of greater than 70 percent to encourage reflection of the germicidal ultraviolet light around the circuitous passageway for interaction with air moving along the circuitous passageway.

15. The unit of claim 14, wherein the housing comprises at least one exterior surface opposing at least one of the interior surfaces facing the circuitous passageway having a reflectivity less than that of the at least one of the interior surfaces.

16. The unit of claim 13, wherein the light barriers are configured to retain at least 85% of the germicidal ultraviolet light inside the housing.

17. The unit of claim 13, further comprising an air filter mounted to the housing in fluid communication with the circuitous passageway,

18. The unit of claim 17, wherein the air filter is arranged at the inlet or the outlet to provide at least a portion of one of the light barriers that discourage egress of the germicidal ultraviolet light from the housing.

19. The unit claim 18, wherein the fan is arranged at the inlet and/or the outlet to provide at least a portion of one of the light barriers that discourage egress of the germicidal ultraviolet light from the housing.

20. A method of operating an over the road vehicle as recited in claim 1, the method including the steps of
operating the climate control system to create a first flow of heated or cooled air moved by the air mover included in the climate control system through vents into the passenger compartment, and
operating the modular air treatment unit independent of the climate control system to create a second flow of air moved by the fan included in the modular air treatment unit flowing directly from the passenger compartment via the inlet and directly to the passenger compartment via the outlet.
